(19) United States
(12) Patent Application Publication
(10) Pub. No.: US 2026/0295186 A1
(43) Pub. Date: Oct. 1, 2026

(54) TRACHEOSTOMY MONITORING SYSTEM

(71) Applicant: The Trustees of the Stevens Institute of Technology, Hoboken, NJ (US)

(72) Inventors: Sally Shady, Upper Saddle River, NJ (US); Kevin Lu, Millington, NJ (US); Frank Castello, Livingston, NJ (US); Sean Pelcher, Berkeley Heights, NJ (US)

(21) Appl. No.: 19/563,559

(22) Filed: Mar. 11, 2026

Related U.S. Application Data
(60) Provisional application No. 63/777,909, filed on Mar. 26, 2025.

(51) Int. Cl. A61M 16/04; A61M 16/00; A61M 16/10

(52) U.S. Cl. CPC A61M 16/0465; A61M 16/0003; A61M 16/0051; A61M 16/024; A61M 16/1045; A61M 2205/18; A61M 2205/3327; A61M 2205/3368; A61M 2205/3375; A61M 2205/3576; A61M 2205/502; A61M 2205/583; A61M 2205/8206; A61M 2205/8237; A61M 2230/42

(57) ABSTRACT

Described herein is a breathing detection device. The device includes a rigid housing comprising a first body and a second body; a first tubular body extending outwards from an exterior of the first body; and a second tubular body extending outwards from an exterior of the second body. Proximal ends of the first tubular body and second tubular body interface with one another to define a channel. The first body defines a cavity sized to house a plurality of electrical components comprising at least a temperature sensor configured to detect a temperature of air passing through the channel, and an audio sensor configured to detect a sound of the air passing through the channel. The first body comprises a temperature sensor mount having protrusions that define a first shape to hold the temperature sensor and that define a second shape to pass temperature sensor wiring into the cavity.

### CROSS-REFERENCE TO RELATED APPLICATIONS

This application claims priority to U.S. patent application No. 63/777,909, filed Mar. 26, 2025, the contents of which are incorporated herein by reference in their entirety.

### BACKGROUND

The trachea is the physiological tube that allows the influx of air from the nose and mouth to reach the lung and is attached to the larynx superiorly, and the main bronchi inferiorly. The trachea is the only pathway that air can enter the body and reach the lungs, but it possesses a relatively small diameter. Because of this, the trachea can easily become obstructed. A tracheostomy procedure is commonly performed when air cannot enter through the nose or mouth due to an upper airway obstruction, spinal cord injury, inability to clear secretion, or other situations requiring long-term mechanical ventilation.

A tracheostomy is performed to create a direct passageway for air through an incision and a tube placed in the throat. Each year, over 100,000 patients in the United States will require a tracheostomy; approximately 5,000 of these cases are pediatric patients. A large number of these patients (i.e., as many as 48%) may experience complications, such as obstructed airways caused by secretions and displacement of the tube. Unfortunately, patients with tracheostomies may have to undergo emergency surgery in the case of an undetected obstruction in the breathing tube leading to oxygen deprivation. Along with mucus and secretions, upper airway obstruction can involve growths and tumors that may be present in the airways of the nose and mouth (located above or within the trachea).

Obstructions routinely form within a tracheostomy tube from mucus secretions, and/or bacterial colonies. Early detection is key to prevent acute symptoms, among which are low oxygen saturation, elevated heart rates, and potential brain damage due to lack of oxygen. Prior devices do not directly measure exhalation and rely on signs of hypoxia to alarm. For example, pulse oximetry alerts when oxygen levels have begun dropping to low levels, meaning the patient has already been without oxygen for an extended period of time. Electrocardiogram (ECG) systems measure secondary effect of oxygen deprivation, and once changes in heart rate/pattern deviate from normal it may be too late to reverse. Early detection can prevent oxygen deprivation and possible brain damage. Oxygen deprivation can lead to brain damage and can quickly become fatal within a time span of minutes. The aforementioned time spans are shortened for pediatric patients, who represent a more vulnerable population due to their inability to communicate when experiencing a life-threatening health complication.

Since occlusion of the tracheostomy tube by secretions can cause serious complications, it is imperative to detect occlusions as early as possible. Patients with tracheostomies requiring mechanical ventilation are monitored by the ventilator for signs of occlusions (i.e., obstructions), which signs include, for example, a sudden increase in ventilation pressure, very low tidal volume of respiratory gases, difficulty providing bag valve mask ventilation and the inability to pass a suction catheter. Paradoxically, as patients improve and are weaned from mechanical ventilation, their risk of death or disability increases due to obstruction of their tracheostomy. Once a patient with a tracheostomy has been removed from mechanical ventilation, there is no way to use the ventilator alarms to monitor the patient. Patients with tracheostomies who are not on mechanical ventilation must rely on secondary measures of safety such as pulse oximetry and/or ECG monitoring, both of which may detect complications after it is too late to intervene.

Traditionally, caretakers of patients with tracheostomies monitor the vital signs of the patient using systems such as pulse oximetry and ECG as customary tools. However, these monitoring systems are limited as they monitor secondary effects of oxygen deprivation. Additionally, these devices work best when the patient is stationary. As motion artifact results in false alarms. For instance, pulse oximetry involves the measurement of absorption of light by hemoglobin resulting in the calculation of an oxygen saturation. Pulse oximetry works best when the subject is not moving as motion artifacts often result in many false alarms. With the pulse oximetry device failing to obtain accurate readings, many false negatives and false positives are received, constituting an inherent lack of accuracy that is present using this system. Another shortcoming is that the pulse oximetry detects when the overall blood oxygen level has dropped significantly, which is a secondary effect of a tracheostomy obstruction or failure. By the time of detection, the blood oxygen level may have dropped significantly, meaning the patient will have been without sufficient oxygen for an extended period of time. Due to the short timeframe (i.e., minutes) available before a pediatric patient experiences brain damage due to oxygen deprivation, doctors have very little time to address the problem by the time a pulse oximeter detects it. In another example, an ECG system monitors a patient's heart rhythm. The resultant waveform of the heartbeat is displayed and if the rhythm is irregular and/or abnormal, the alarm will sound. This, again, only monitors the secondary effects of a tracheostomy obstruction or failure. When blood oxygen levels have dropped, the heart rate is then affected. Therefore, the ECG gives very little time for the doctors to react to the drop in the oxygen levels in the blood.

Other conventional respiratory rate monitors may be used in treatment of other conditions, such as obstructive sleep apnea. However, these monitors generally require the patient to wear an oxygen mask over the patient's nose and mouth. Further, tubing that provides oxygen to the mask and wiring connected to sensors on the mask that detect a respiratory rate generally create a cluster of objects around the neck and face area of the patient. As such, the mask secured around a patient's head, as well as the cluster of tubing and wiring, impedes that ability of a healthcare practitioner to treat the patient.

### SUMMARY

The present disclosure relates generally to tracheostomy devices, and more particularly, to tracheostomy monitoring systems for detecting abnormal breathing.

In one or more aspects, the disclosed technology relates to a breathing detection device. The device includes a rigid housing comprising a first body and a second body coupled to one another; a first tubular body extending outwards from an exterior of the first body; and a second tubular body extending outwards from an exterior of the second body, wherein proximal ends of the first tubular body and second tubular body interface with one another to define a channel that extends across the housing and is sized to pass air therethrough, wherein the first body defines a cavity sized to house a plurality of electrical components comprising at least a temperature sensor configured to detect a temperature of air passing through the channel, and an audio sensor configured to detect a sound of the air passing through the channel, and wherein the first body comprises a temperature sensor mount having protrusions that define a first shape to hold the temperature sensor therein such that the temperature sensor interfaces with the channel and that define a second shape to pass temperature sensor wiring into the cavity.

In one or more aspects, the disclosed technology relates to tracheostomy monitoring system. The tracheostomy monitoring system includes a breathing detection device. The breathing detection device comprises a rigid housing comprising a first body and a second body coupled to one another, a first tubular body extending outwards from an exterior of the first body, and a second tubular body extending outwards from an exterior of the second body, wherein proximal ends of the first tubular body and second tubular body interface with one another to define a channel that extends across the housing and is sized to pass air therethrough, wherein the first body defines a cavity sized to house a plurality of electrical components comprising at least a microcontroller, a temperature sensor configured to detect a temperature of air passing through the channel, an audio sensor configured to detect a sound of the air passing through the channel, and a transceiver configured to transmit to a computing device temperature data and audio data associated with air passing through the channel, and wherein the first body comprises a temperature sensor mount having protrusions that define a first shape to hold the temperature sensor therein such that the temperature sensor interfaces with the channel and that define a second shape to pass temperature sensor wiring into the cavity. The tracheostomy monitoring system includes a computing device comprising a memory storing instructions, a communications module, and at least one processor coupled to the communications module and the memory. The at least one processor configured to execute the instructions to receive temperature data and audio data associated with air passing through the channel of the breathing detection device, determine a breath per minute (BPM) associated with each of the received temperature data and the audio data, provide and display a first alert on the computing device based on the BPM associated with the temperature data and the audio data being outside a predetermined BPM range for a first threshold period of time, provide and display a second alert on the computing device based on the BPM associated with the temperature data and the audio data being zero for a second threshold period of time, and provide and display a third alert on the computing device based on the temperature data indicating a decrease in temperature for a third threshold period of time.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### BRIEF DESCRIPTION OF THE DRAWINGS

The following drawings are illustrative of particular embodiments of the present disclosure and therefore do not limit the scope of the present disclosure. The drawings are not to scale and are intended for use in conjunction with the explanations in the following detailed description.

FIG. 1A is a perspective view of an example breathing detection device. FIGS. 1B-1E illustrate various views of the portions of the example breathing detection device.

FIG. 2 is a diagram illustrating an example tracheostomy monitoring system.

FIG. 3 is an example flowchart that illustrates a process of detecting abnormal breathing.

FIG. 4 is a block diagram depicting components of a data processing system.

### DETAILED DESCRIPTION

The following discussion omits or only briefly describes conventional features of tracheostomy devices that are apparent to those skilled in the art. It is noted that various embodiments are described in detail with reference to the drawings, in which like reference numerals represent like parts and assemblies throughout the several views. Reference to various embodiments does not limit the scope of the claims attached hereto. Additionally, any examples set forth in this specification are intended to be non-limiting and merely set forth some of the many possible embodiments for the appended claims. Further, particular features described herein can be used in combination with other described features in each of the various possible combinations and permutations.

Unless otherwise specifically defined herein, all terms are to be given their broadest reasonable interpretation including meanings implied from the specification as well as meanings understood by those skilled in the art and/or as defined in dictionaries, treatises, etc. It is noted that, as used in the specification and the appended claims, the singular forms “a,” “an” and “the” include plural referents unless otherwise specified, and that the terms “includes” and/or “including,” when used in this specification, specify the presence of stated features, elements, and/or components, but do not preclude the presence or addition of one or more other features, steps, operations, elements, components, and/or groups thereof.

Relative terms such as “horizontal,” “vertical,” “up,” “down,” “top,” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively or operably connected” is such an attachment, coupling or connection that allows the pertinent structures to operate as intended by virtue of that relationship.

Reference throughout the specification to “one embodiment”, “an embodiment” or “some embodiments” means that a particular feature, structure, or characteristic described in connection with an embodiment is included in at least one embodiment of the subject matter disclosed. Thus, the appearance of the phrases “in one embodiment”, “in an embodiment” or “in some embodiments” in various places throughout the specification is not necessarily referring to the same embodiment. Further, the particular features, structures or characteristics of “one embodiment”, “an embodiment” or “some embodiments” may be combined in any suitable manner with each other to form additional embodiments of such combinations. It is intended that embodiments of the disclosed subject matter cover modifications and variations thereof. Terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Moreover, throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range. As used herein, the term “about” in reference to a measurable value, such as an amount, a temporal duration, and the like, is meant to encompass the specified value and/or variations of plus or minus 20%, plus or minus 10%, plus or minus 5%, plus or minus 1%, and plus or minus 0.1% of the specified value, as such variations are appropriate.

The terms “proximal,” “distal,” “anterior,” “posterior,” “medial,” “lateral,” “superior,” and “inferior” are defined by their standard usage indicating a directional term of reference. For example, “proximal” refers to a position that is situated nearer to the center of a body or point of attachment, while “distal” refers to a position that is situated away from the center of the body or point of attachment. In another example, “anterior” refers to the front of a body or structure, while “posterior” refers to the rear of a body or structure. In another example, “medial” refers to the direction towards the midline of a body or structure, and “lateral” refers to the direction away from the midline of a body or structure. In some examples, “lateral” or “laterally” may refer to any sideways direction. In another example, “superior” refers to the top of a body or structure, while “inferior” refers to the bottom of a body or structure. It should be understood, however, that the directional term of reference may be interpreted within the context of a specific body or structure, such that a directional term referring to a location in the context of the reference body or structure may remain consistent as the orientation of the body or structure changes.

The terms “patient,” “subject,” “individual,” and the like are used interchangeably herein, and refer to any animal amenable to the systems, devices, and methods described herein. The patient, subject or individual may be a mammal, for example, a human.

Conventional tracheostomy devices, such as pulse oximeters and heart rate monitors, determine a respiration rate of a patient by monitoring secondary effects of oxygen deprivation. As such, these devices are inherently delayed in providing notifications that, for example, indicate obstructions or blockages within a tracheostomy tube. This delay may result in significant morbidity and overall increased mortality rates among tracheostomy patients. The embodiments described herein determine respiration rates using the breathing pattern of the patient by directly monitoring air flow through the tracheostomy tube, and thus allows for early detection of abnormal breathing, dislodgement, and obstruction. Moreover, the embodiments described herein provide a compact tracheostomy monitoring device, which reduces cluster around the neck area of the tracheostomy patient.

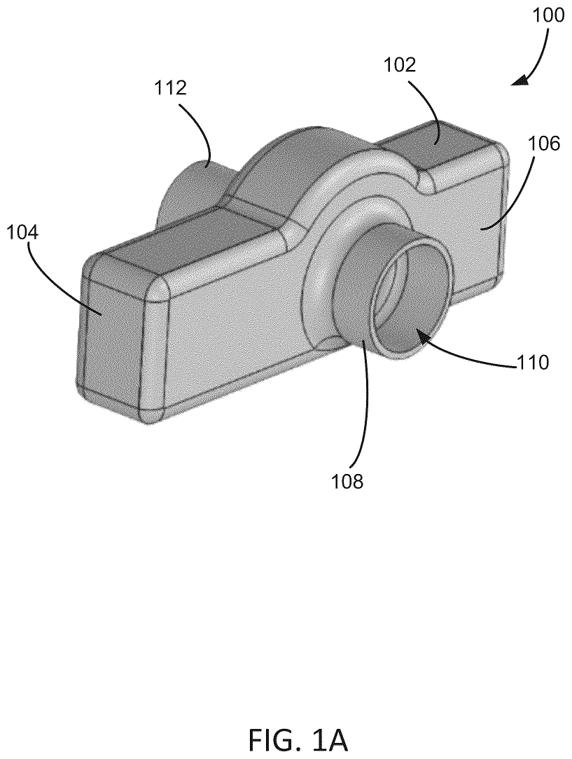

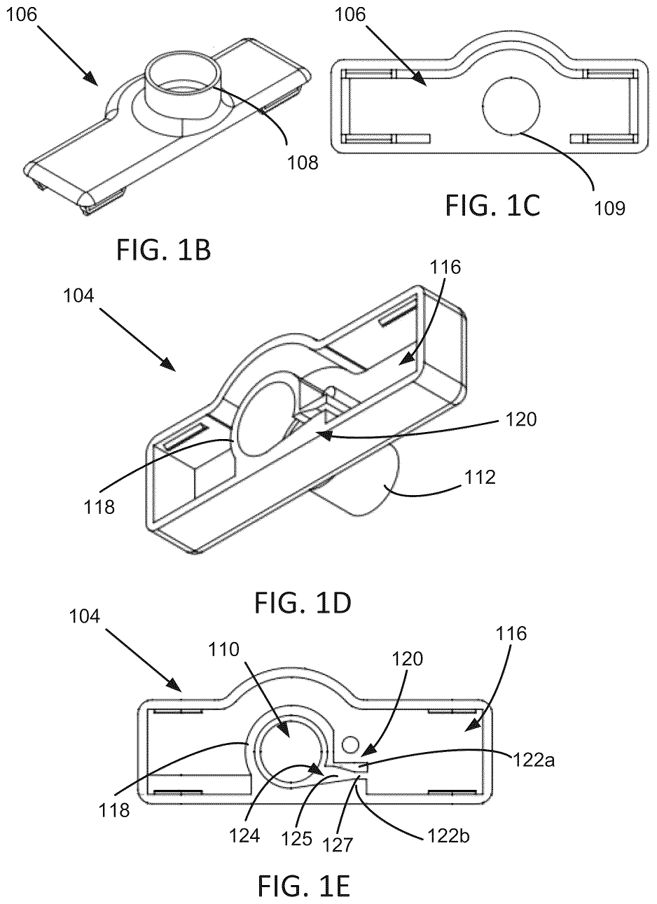

FIG. 1A is a perspective view of an example breathing detection device (hereinafter “device 100”). FIGS. 1B-1E illustrate various views of the portions of the device 100. The device 100 includes a rigid housing 102 defining a cavity 116 therein and a channel 110 extending therethrough. It is noted that the examples provided herein describe the device 100 being coupled with tracheostomy tubes and other conventional tracheostomy attachments to detect breathing issues; however, it should be understood that the device 100 may be used to monitor other conditions that result from obstructed airways. Further, the device 100 may be integrated with other biomedical sensors to capture respiration rates.

The housing 102 may be formed using one or a variety of manufacturing processes, such as, but not limited to, injection molding, 3D printing, and the like. The housing 102 may be formed of material, such as, but not limited to, thermoplastics (e.g., acrylonitrile butadiene styrene (ABS)), polylactic acid (PLA) nylon, polypropylene, polyethylene, polycarbonate, polyvinyl chloride (PVC), polyvinylidene fluoride (PVDF), metal suitable for medical applications (e.g., surgical steel), and other like materials. The housing 102 may be an assembly of separate component parts formed from, for example, a first body 104 and a second body 106. The first body 104 and second body 106 may be coupled to one another. For example, the first body 104 and second body 106 may be fastened to one another by being screwed, snapped, or bonded (e.g., using welding or adhesive) to one another thereby enclosing the cavity 116 to protect electronic components housed therein.

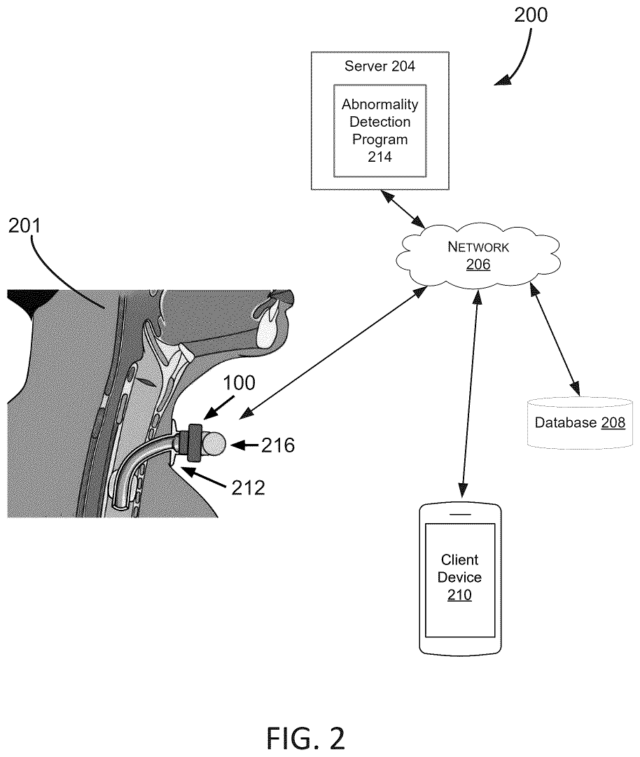

Tubular bodies 108 and 112 may be disposed on opposing sides of the housing 102. For example, the tubular body 112 may be disposed on the exterior of the first body 104 and extend outwards from the housing 102. The tubular body 108 may be disposed on the exterior of the second body 106 and extend outwards from the housing 102. The tubular bodies 108 and 112 may be aligned with one another forming the channel 110 that extends through the housing 102. In one or more cases, when the first body 104 and the second body 106 are coupled to one another, the proximal end 108 of tubular body 108 and the proximal end 118 of the tubular body 108 may interface with one another to form the channel 110 extending across the housing 102. The tubular bodies 108 and 112 may each be sized to couple to tubing, such as, but not limited to tracheostomy tubes (e.g., the tracheostomy tube 212 illustrated in FIG. 2) and other conventional attachments, such as a heat and moisture exchanger (HME) attachment (e.g., the HME 216 illustrated in FIG. 2) configured to reduce secretions or a Passy-Muit valve attachment that allows a patient to speak. The tubular bodies 108 and 112 may have either a male or female end configured to mate with the tubing or attachments, for example, by being press fit to the tubing or attachments. For instance, the tubular body 108 may have a female end sized to fit within an opening of the HME attachment 216. In another instance, the tubular body 112 may have a male end sized to fit over an opening of the tracheostomy tube 212. In yet other cases, the tubular body 108 may be coupled to a tracheostomy tube adapter for use with pediatric patients.

The first body 104 may define a volume of the cavity 116, in which the second body 106 acts as a lid when coupled with the first body 104. The cavity 116 may be sized to house one or more electrical components. For instance, the device 100 may include one or more of a microcontroller, a power source, one or more microphones, a temperature sensor, and a transceiver operably coupled to one another within the housing 102. For example, the power source, microphone, temperature sensor and transceiver may be coupled to the microcontroller via wiring soldered to the respective inputs and outputs of the microcontroller. The microcontroller may be, for example, an Arduino Seeed XIAO BLE Sense nRF52840 microcontroller. In some cases, the microphone is included on the microcontroller. For example, the microphone may be mounted on and electrically coupled with the microcontroller. In other cases, the microphone is separate from but electrically coupled with the microcontroller. For example, the microphone may be electrically coupled with the microcontroller and may be mounted in the housing 102 in an area separate from the microcontroller. The power source may be a battery, such as a 3.7 V 150 mAh lithium polymer battery. In one or more cases, the battery is rechargeable. In some cases, the second body 106 may be removed from the first body 104 to access and remove the battery from the microcontroller to recharge the battery. In other cases, a charging port is operably coupled with the battery and disposed on a wall of the second body 106. As such, a user may plug a power source into the charging port to charge the battery without accessing the cavity 116 of the housing 102. Moreover, as the battery may be charged without accessing the cavity 116, the device 100 may remain operably coupled to a patient (e.g., via the tracheostomy tube 212 and HME attachment 216) and maintain functionality.

The temperature sensor may be, for example, a thermistor. For instance, the thermistor may be an Adafruit 10K precision epoxy thermistor. The temperature sensor may be positioned within the housing 102 to contact airflow passing through the channel 110 and capture temperature readings of the airflow. In one or more cases, the housing 102 includes a sensor mount 120 configured to hold the temperature sensor in a fixed position. The sensor mount 120 may be integrated with a portion of the tubular body 112, such that an opening 124 of the sensor mount 120 interfaces with the area defined by the channel 110. The sensor mount 120 may include protrusions 122a, 122b that define the opening 124 of the sensor mount 120. The opening 124 may extend from the channel 110 to the interior of the cavity 116. In some cases, the opening 124 may be formed in the shape of a bottleneck. For instance, the protrusions 122a, 122b may include a portion 125 in which the protrusions 122a, 122b taper from the channel 110 towards one another. Adjacent to the portion 125, the protrusions 122a, 122b form another potion 127 of the opening 124, such that the protrusions 122a, 122b extend parallel with one another from the tapered portion 125 to the cavity 116. In one or more cases, the temperature sensor may be positioned within the portion 125 of the opening 124, and wiring may pass from the temperature sensor to the microcontroller through the portion 127 of the opening 124. In some cases, the portion 127 may be sealed around the wiring passing therethrough to prevent airflow or other fluids from entering the cavity 116.

In one or more cases, the microphone may be configured to obtain and provide audio measurements to the microcontroller. The microphone may capture the sound of airflow passing through the channel 110. The captured sound may be provided as audio measurements to the microcontroller. In some cases, the microphone is built into the board of the microcontroller. The transceiver is configured to transmit and receive signals (e.g., audio measurement data and temperature data) from the microcontroller to an external device (e.g., client device 210 illustrated in FIG. 2). In some cases, the transceiver is a Bluetooth® transceiver and may be paired with a remote computing device, such as client device 110, to send and receive data.

FIG. 2 is a diagram illustrating an example tracheostomy monitoring environment 200. FIG. 2 provides only an illustration of one implementation and does not imply any limitations with regard to the environment in which different embodiments may be implemented. Many modifications to the depicted environment may be made by those skilled in the art without departing from the scope of the embodiments of the present disclosure. The tracheostomy monitoring environment 200 includes a network 206, a server 204 that operates an abnormality detection program 214, a database 208, device 100, and one or more computing devices, such as a client device 110. Further, the device 100 may be coupled with the tracheostomy tube 212 inserted into a patient 201 and the HME attachment 216.

The network 206 interconnects the server 204, database 208, the client device 210, and the device 100. In general, the network 106 can be any combination of connections and protocols capable of supporting communications between the server 204, the client device 210, the device 100, the database 208, and the program 214. The network 206 can include, for example, one or more of wired Ethernet, fiber optic connections, wireless connections including any of the various 802.11 standards, cellular WAN infrastructures such as, but not limited to, 3G, 4G/LTE, or 5G networks, Bluetooth®, Bluetooth® Low Energy (BLE) or Zigbee® communication links, or any other method by which an electronic device is capable of communicating with another. In some cases, the network 206 may be implemented over one or more of a Virtual Private Network (VPN), a local area network (LAN), a telecommunications network, a wide area network (WAN), such as the Internet, a virtual local area network (VLAN), and the like. The network 206 can also include wire cables, wireless communication links, fiber optic cables, routers, switches and/or firewalls.

The server 204 is a web-based server hosting the program 214. In one or more cases, the server 204 can be a web server, a blade server, a computer including one or more processors and at least one non-transitory computer readable memory, a mobile computing device, a laptop computer, a tablet computer, a netbook computer, a personal computer (PC), a desktop computer, or any programmable electronic device or computing system capable of receiving and sending data, via the network 206, and performing computer-readable program instructions. In one or more cases, the server 204 can be a data center, consisting of a collection of networks and servers providing an IT service, such as virtual servers and applications deployed on virtual servers, to an external party. In one or more cases, the server 204 represents a computing system utilizing clustered computers and components (e.g., database server computer, application server computers, etc.) that act as a single pool of seamless resources, such as in a cloud computing environment when accessed within the environment 200.

In one or more cases, the server 204 includes the database 208 for storing data including, but not limited to, audio measurement data, temperature data, and respiration rate limits. The database 208 can be one of, a web server, a mobile computing device, a laptop computer, a tablet computer, a netbook computer, a personal computer (PC), a desktop computer, or any programmable electronic device or computing system capable of receiving, storing, and sending data, and performing computer readable program instructions capable of communicating with the server 204, the client device 210, and the scanning device 100, via the network 206. In one or more cases, the database 208 can represent virtual instances operating on a computing system utilizing clustered computers and components (e.g., database server computer, application server computers, etc.) that act as a single pool of seamless resources when accessed within the environment 200.

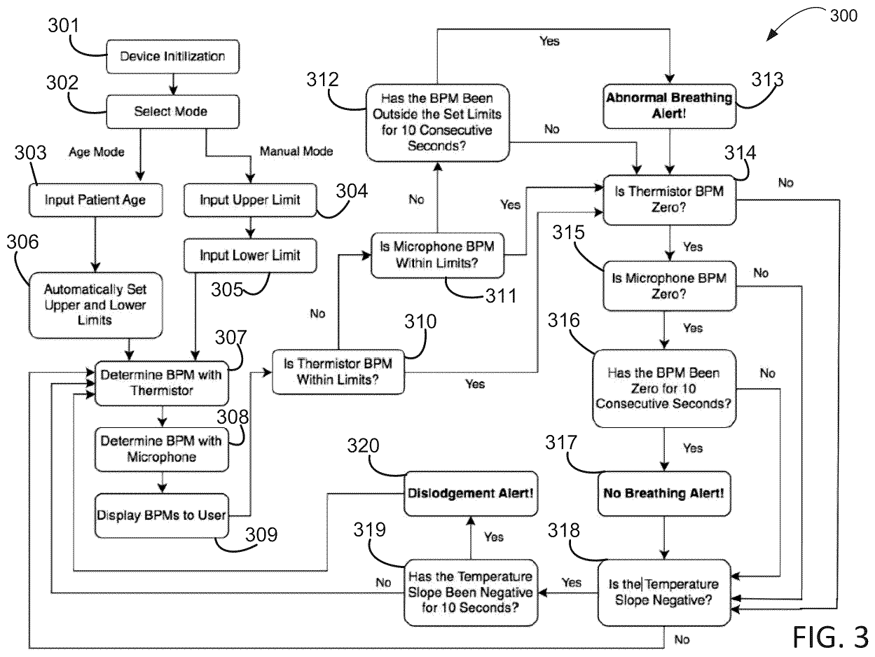

In one or more cases, the abnormality detection program 114 is configured to perform operations to detect breathing abnormalities in real-time. For example, the abnormality detection program 114 may implement one or more operations of process 300 to determine characteristics of airflow passing through the device 100 to detect breathing abnormalities in real-time, as illustrated in FIG. 3. The program 214 operates on a central server, such as the server 204, and may be utilized by one or more computing electronic devices, such as client device 210 and device 100, via an application downloaded from the central server or a third-party application store and executed on the one or more computing electronic devices. In one or more cases, the program 214 may be a software-based program, downloaded from a central server, such as the server 204, and installed on one or more computing electronic devices, such as client device 210. In one or more cases, the program 214 can be utilized as a software service provided by a third-party cloud service provider (not shown). In one or more cases, the program 214 may be preinstalled, as software and/or firmware, on the one or more computing electronic devices. In one or more cases, program 214 may be installed onto the one or more computing electronic devices via an external storage device, such as a universal serial bus (USB) flash drive. In one or more cases, software executing instructions of the program 214 may be stored on a non-transitory computer-readable medium. The software performs some or all of the instructions when executed by one or more processors. Further, one or more aspects of the disclosure relate to algorithms executed in computer software. Though certain aspects may be described in particular programming languages, or executed on particular operating systems or computing platforms, it is understood that the systems and methods described herein are not limited to any particular computing language, platform, or combination thereof. For example, software executing the algorithms described herein may be written in any programming language, compiled or interpreted, including but not limited to C, C++, C #, Objective-C, Java, JavaScript, Python, PHP, Perl, Ruby, or Visual Basic.

In one or more cases, the client device 210 is an electronic computing device, such as a desktop computer, a laptop computer, a tablet computer, a personal digital assistant (PDA), a smart phone, a thin client, or any other electronic device or computing system capable of communicating with the server 204 through the network 206. The client device 210 may be a client to the server 204. In other cases, the client device 210 can be any suitable type of mobile device capable of running mobile applications, including smart phones, tablets, slate, or any type of device that runs a mobile operating system. For example, the client device 210 may be a mobile device operated by a user, such as a healthcare provider, and capable of connecting to a network, such as the network 206, to receive audio measurement data and temperature data from the device 100 and transmit the data to the program 214.

In one or more cases, the client device 210 can include a user interface for providing an end user with the capability to interact with the program 214. A user interface refers to the information (such as graphics, text, and sound) the program 214 presents to a user. A user interface can be, for example, a keyboard that allows a user to input text, a touchscreen that accepts input from a user via touch of a body part and/or a stylus, or the like. A user may access the program 214 through the user interface to enable the program 214 to operate on the user's device.

FIG. 3 is an example flowchart that illustrates a process 300 of detecting abnormal breathing. The device 100 is initialized (at 301). In some cases, the device 100 is initialized by being powered on and connecting to the client device 210 via, for example, a wireless connection. In some cases, the device 100 is initialized before being attached to the tracheostomy tube 212 and the HME attachment 216. In other cases, the device 100 is initialized after being attached to the tracheostomy tube 212 and the HME attachment 216. In yet other cases, the device 100 is attached to the tracheostomy tube 212 and the HME attachment 216 after the respiration rate limits are set, as further described herein. An application associated with the abnormality detection program 214 may be launched on the client device 210. The program 214 displays one or more modes on the user interface. For example, the modes may be an age mode and a manual mode.

The program 214 receives a selection of a mode (at 302). For example, a healthcare provider may input a selection of a mode via a user interface displayed on the client device 210. For the cases in which the program 214 receives a selection of age mode, a user inputs the age of the patient, and the program 214 automatically sets upper and lower respiration rate limits (at 306). The upper and lower respiration rate limits may be based on pre-existing data. For the cases in which the program 214 receives a selection of manual mode, the user inputs upper respiration rate limits (at 304) and lower respiration rate limits (at 305).

Having set the respiration rate limits, the device 100 may collect audio measurement data and temperature data based on the airflow passing through the channel 110 of the device 100. For example, as the user's breath passes through the channel 110 of the device 100, the device 100 captures audio measurement data via the microphone and temperature data via the temperature sensor within the device 100. The device 100 provides (e.g., via wireless transmission) the captured audio measurement data and temperature data to the program 214 via the client device 210. Having received the captured data, the program 214 determines the breaths per minute (BPM) of the patient. For example the program 214 determines the BPM of the patient based on the temperature data (at 307). In another example, the program 214 determines the BPM of the patient based on the audio measurement data (at 308). It is noted that the program 214 determines temperature data from the temperature sensor and audio measurement data from the microphone in a same or similar manner as those processes described in U.S. patent application Ser. No. 16/938,876. For instance, the program 214 utilizes Steinhart-Hart equation models to determine temperature data. As such, U.S. patent application Ser. No. 16/938,876 is incorporated herein by reference in its entirety.

In one or more cases, the program 214 displays the BPM based on the temperature data and the BPM based on the audio data on the user interface of the client device 210 (at 309). In some cases, the program 214 determines whether the BPM based on the temperature data is within the set respiration rate limits (at 310). For the cases in which the program 214 determines that the BPM based on the temperature data is not within the set respiration rate limits (310:NO), the program determines whether the BPM based on the audio data is within the set respiration rate limits (at 311). For the cases in which the program 214 determines that the BPM based on the audio data is not within the set respiration rate limits (311:NO), the program determines whether the BPM associated with the temperature data and/or audio data is outside the set respiration rate limits for a threshold period of time (e.g., 10 seconds) (at 312). For the cases, in which the program 214 determines that the BPM associated with the temperature data and/or audio data is outside the set respiration rate limits for the threshold period of time (312:YES), the program 214 determines that the patient is exhibiting symptoms of abnormal breathing patterns and provides an abnormal breathing alert to the client device 210 (at 313). For example, the program 214 may display the abnormal breathing alert on the user interface of the client device 210. In another example, the program 214 may provide and display the abnormal breathing alert on one or more devices associated with a nurses station at the healthcare facility of the patient. Thus, for the cases in which the BPM associated with the temperature data and audio data are outside the set respiration rate limits for the threshold period of time, the abnormal breathing alert is provided to one or more devices until the issue is resolved.

For the cases in which the program 214 determines that the BPM based on the temperature data is within the set respiration rate limits (310:YES), determines that the BPM based on the temperature data is not within the set respiration rate limits (310:NO) and that the BPM based on the audio data is within the set respiration rate limits (311:YES), or that the BPM based on the temperature data and the audio data (310:NO, and 311:NO) and that the BPM associated with the temperature data and/or audio data is not outside the set respiration rate limits for the threshold period of time (312:NO), the program 214 determines whether the BPM based on the temperature data is zero (at 314). Further, in some cases that provided an abnormal breathing alert (at 313), the program 214 may continue to determine whether the BPM based on the temperature data is zero (at 314).

For the cases in which the program 214 determines that BPM based on the temperature data is zero (314:YES), the program 214 determines whether the BPM based on the audio data is zero (at 315). For the cases in which the program 214 determines that the BPM based on the audio data is zero (315:YES), the program 214 determines whether the BPM associated with the temperature data and/or audio data is zero for a threshold period of time (e.g., 10 seconds) (at 316). For the cases in which the program 214 determines that the BPM associated with the temperature data and/or audio data is zero for the threshold period of time (316:YES), the program 214 determines that the patient is exhibiting symptoms of not breathing and provides a cessation of breathing alert to the client device 210 (at 317). For example, the program 214 may display the no breathing alert on the user interface of the client device 210. In another example, the program 214 may provide and display the no breathing alert on one or more devices associated with the nurses station at the healthcare facility of the patient. Thus, for the cases in which the BPM associated with the temperature data and audio data are zero for the threshold period of time, the no breathing alert is provided to one or more devices until the issue is resolved.

For the cases in which the program 214 determines that BPM based on the temperature data is not zero (314:NO), that the BPM based on the temperature data is zero (314:YES) and the BPM based on the audio data is not zero (315:NO), or that the BPM based on the temperature data and audio data are zero (314:YES, and 315:YES) and the BPM is not zero for the threshold period of time (316:NO), the program 214 determines whether the temperature slope is negative (at 318). Further, in some cases that provided a cessation of breathing alert (at 317), the program 214 may continue to determine whether the temperature slope is negative (at 318). For the cases in which the program 214 determines that the temperature slope is not negative (318:NO), the program 214 returns to determining the BPM based on the temperature data (at 307).

For the cases in which the program 214 determines that the temperature slope is negative (318:YES), the program 214 determines whether the temperature slope has been negative for a threshold period of time (e.g., 10 seconds) (at 319). For the cases in which the program 214 determines that the temperature slope has not been negative for a threshold period of time (319:NO), the program 214 determines that the device 100 and/or the tracheostomy tube 212 has become dislodged from the patient and provides an abnormal breathing alert to the client device 210 (at 320). For example, the program 214 may display the dislodgement alert on the user interface of the client device 210. In another example, the program 214 may provide and display the dislodgement alert on one or more devices associated with a nurses station at the healthcare facility of the patient. Thus, for the cases in which the temperature data is declining (i.e., the measured temperature of the device 100 is cooling) for a threshold period of time, the dislodgement alert is provided to one or more devices until the issue is resolved.

In one or more cases, the device 100 is configured for the continuous, noninvasive, and wireless monitoring of respiration rate within the pediatric and adult tracheostomy patient populations. The device 100 and program 214 may be used caregivers in hospitals, hospital-type environments, such as long-term care facilities and home settings, and mobile and home healthcare environments. In one or more cases, the device 100 and program 214 may implement one or more artificial intelligence models to detect patient specific breathing patterns unique to the physiology of the patient. For example, the device 100 and program 214 may implement the one or more artificial intelligence models to detect, for example, but not limited to, asthma, Chronic Obstructive Pulmonary Disease, pneumonia, blockage in the tracheostomy tube, and the like. The device 100 and program 214 may implement the one or more artificial intelligence models to detect, for example, breathing rates based on temperature and audio data, a dislodgement of the tracheostomy tube based on temperature and audio data, and an obstruction or a degree of obstruction within the tracheostomy tube based on audio data. The patient specific breathing patterns may be used to set upper and lower respiration limits for use in process 300 as described herein. As such, the program 214 may provide alerts as described herein based on detecting deviations from the patient specific breathing patterns. Furthermore, the housing and multi-sensory system of the device 100 enables the device 100 to wirelessly operate and monitor breathing patterns directly from the airflow of the tracheostomy patient. Moreover, by monitoring airflow temperature and sound, the device 100 and program 214 reduce the number of false positives and negatives when detecting breathing issues. The device 100 and program 214 additionally provide caregivers with real-time data on the status of a tracheostomy tube. In pediatric cases, the device 100 and program 214 provide early detection of tracheostomy issues, which is critical as hypoxia can occur within a few minutes due to the unique physiology of pediatric patients.

The tracheostomy device was tested by attaching the tracheostomy device to an ASL 5000 Breathing Simulator, along with an HME device, to determine whether the thermistor and microphone could detect breaths and accurately calculate the respiration rate for the adult population. In the simulation, a healthy adult patient model was employed with a tidal volume of 500 mL using the settings of the ASL 5000 Breathing Simulator. The respiration rate was then kept constant at 10 breaths per minute while the test ran for five minutes. Afterwards, data was gathered from both the tracheostomy device and the ASL 5000 Breathing Simulator and compared to determine whether the peaks (representing breaths) matched, which would indicate that the tracheostomy device was functioning as intended. This was repeated at 20, 30, and 40 breaths per minute, and the results proved that the tracheostomy device was accurately capturing the breaths from the ASL 5000 Breathing Simulator. Based on the results, it appeared that the higher the breaths per minute, the more accurate the device was at capturing the true respiration rate. The temperature sensor was also used to verify these results and its ability to detect breathing rates.

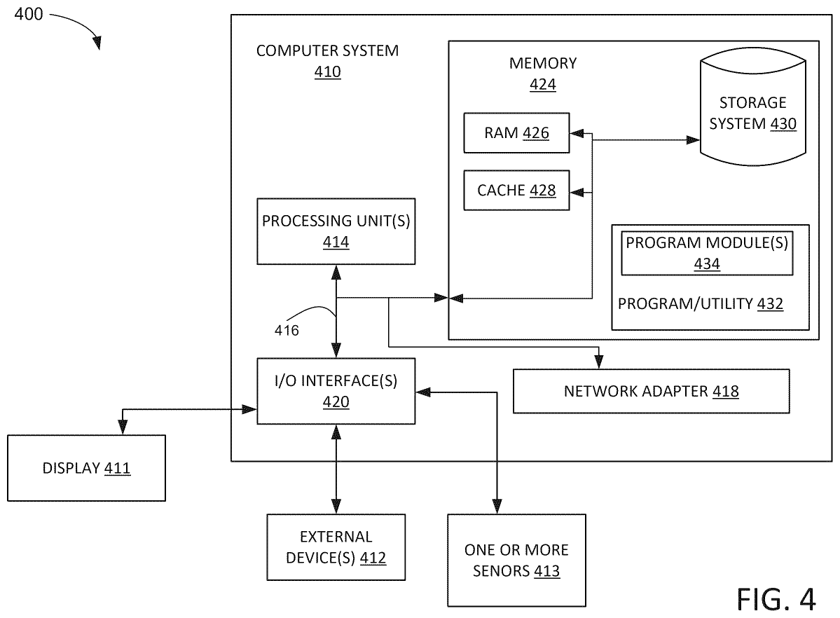

FIG. 4 is a block diagram depicting components of a data processing system. FIG. 4 is a block diagram, generally designated 400, depicting components of computing device capable of operating, for example, the program 214 in accordance with embodiments of the present disclosure. FIG. 4 provides only an illustration of one implementation and does not imply any limitations with regard to the environment in that different embodiments may be implemented. Many modifications to the depicted environment may be made.

In one or more cases, a computing system, such as server 104, client device 110, and/or scanning device 116 is shown in the form of a general-purpose computing device, such as computer system 410. The components of the computer system 410 may include, but are not limited to, one or more processors or processing unit 414, a memory 424, and a bus 416 that couples various system components including the memory 424 to the processing unit 414.

The bus 416 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

The computer system 410 typically includes a variety of computer system readable media. Such media may be any available media that is accessible by the computer system 410, and it includes both volatile and non-volatile media, removable and non-removable media.

The memory 424 can include computer system readable media in the form of volatile memory, such as random access memory (RAM) 426 and/or cache memory 428. The computer system 410 may further include other removable/non-removable, volatile/non-volatile computer system storage media. By way of example only, the storage system 430 can be provided for reading from and writing to a non-removable, non-volatile media, for example, magnetic media (not shown and typically called a “hard drive”), and/or solid-state non-volatile media, for example flash memory. Although not shown, a magnetic disk drive for reading from and writing to a removable, non-volatile magnetic disk, a drive for reading from and writing to a removable, non-volatile solid-state disk (e.g., a flash drive), and/or an optical disk drive for reading from or writing to a removable, non-volatile optical disk such as a CD-ROM, DVD-ROM, or other optical media can be provided. In such instances, each can be connected to the bus 416 by one or more data media interfaces. As will be further depicted and described below, the memory 424 may include at least one computer program product having a set (e.g., at least one) of program modules that are configured to carry out the functions of embodiments.

A program/utility 432, having one or more sets of program modules 434, may be stored in the memory 424 by way of example, and not limitation, as well as an operating system, one or more application programs, other program modules, and program data. Each of the operating systems, one or more application programs, other program modules, and program data, or some combination thereof, may include an implementation of a networking environment. The program modules 434 generally carry out the functions and/or methodologies of embodiments as described herein. The computer system 410 may also communicate with one or more external device(s) 412 such as a keyboard, a pointing device, a display 411, etc., or one or more devices that enable a user to interact with the computer system 410 and any devices (e.g., a network adapter, modem, wireless network adapter, Bluetooth adapter, etc.) that enable the computer system 410 to communicate with one or more other computing devices. Such communication can occur via Input/Output (I/O) interface(s) 420. Still yet, the computer system 410 can communicate with one or more networks such as a local area network (LAN), a general wide area network (WAN), and/or a public network (e.g., the Internet) via a network adapter 418. As depicted, the network adapter 418 communicates with the other components of the computer system 410 via the bus 416. It should be understood that although not shown, other hardware and software components, such as microcode, device drivers, redundant processing units, external disk drive arrays, RAID systems, tape drives, and data archival storage systems may be used in conjunction with the computer system 410.

The embodiments described in the present disclosure may relate to a system, a method, and/or a computer program product. The computer program product may include a computer readable storage medium (or media) having computer readable program instructions for causing a processor to carry out aspects of the embodiments. The computer system 410 also may be operably coupled to one or more sensors 413, such as, but not limited to, a tactile sensor and/or a light sensor.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions, cloud storage, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a non-transitory computer readable storage medium or to an external computer or external storage device via a network, for example, the Internet, a local area network, a wide area network and/or a wireless network. The network may include copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

The computer readable program instructions for carrying out operations of the embodiments may be instruction-set-architecture (ISA) instructions, machine instructions, machine dependent instructions, microcode, firmware instructions, state-setting data, or either source code or object code written in any combination of one or more programming languages, including an object oriented programming language such as C++ and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In one or more cases, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the embodiments described in the present disclosure.

Aspects of the embodiments are described herein with reference to illustrations and/or block diagrams of methods, apparatus (systems), and computer program products. It will be understood that each block of the illustrations and/or block diagrams, and combinations of functions in the illustrations and/or block diagrams, can be implemented by non-transitory computer readable program instructions.

These computer readable program instructions may be provided to a processor of a general-purpose computer, a special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified herein.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified herein (e.g., to control an orientation of a drive wheel and/or position of a drive assembly).

The figures illustrate the architecture, functionality, and operation of possible implementations of systems, methods, and computer program products according to various embodiments. In this regard, each block in the block diagrams may represent a module, segment, or portion of instructions, which comprises one or more executable instructions for implementing the specified logical function(s). In some alternative implementations, the functions noted in the block may occur out of the order noted in the Figures. For example, two blocks shown in succession may be executed substantially concurrently, the blocks may sometimes be executed in the reverse order, depending upon the functionality involved, or the blocks may sometimes be executed out of order, depending upon the functionality involved. It will also be noted that each block of the block diagrams and combinations of blocks in the block diagrams can be implemented by special purpose hardware-based systems that perform the specified functions or acts or carry out combinations of special purpose hardware and computer instructions.

The various embodiments described above are provided by way of illustration only and should not be construed to limit the claims attached hereto. Those skilled in the art will readily recognize various modifications and changes that may be made without following the example embodiments and applications illustrated and described herein, and without departing from the spirit and scope of the following claims.

What is claimed is:

1. A breathing detection device, comprising:
a rigid housing comprising a first body and a second body coupled to one another;
a first tubular body extending outwards from an exterior of the first body; and
a second tubular body extending outwards from an exterior of the second body,
wherein proximal ends of the first tubular body and second tubular body interface with one another to define a channel that extends across the housing and is sized to pass air therethrough,
wherein the first body defines a cavity sized to house a plurality of electrical components comprising at least a temperature sensor configured to detect a temperature of air passing through the channel, and an audio sensor configured to detect a sound of the air passing through the channel, and
wherein the first body comprises a temperature sensor mount having protrusions that define a first shape to hold the temperature sensor therein such that the temperature sensor interfaces with the channel and that define a second shape to pass temperature sensor wiring into the cavity.

2. The breathing detection device of claim 1, wherein the second body comprises a removably lid that encloses the cavity of the first body.

3. The breathing detection device of claim 1, wherein a length of the second tubular body is smaller than a length of the first tubular body.

4. The breathing detection device of claim 1, wherein the second tubular body is sized to fit within an opening of a heat and moisture exchanger attachment or a Passy-Muit valve attachment.

5. The breathing detection device of claim 1, wherein the first tubular body is sized to fit over an opening of a tracheostomy tube or a pediatric tracheostomy tube adapter.

6. The breathing detection device of claim 1, wherein the plurality of electrical components further comprise a microcontroller, a power source, and a transceiver configured to transmit to a user device temperature data and audio data associated with air passing through the channel.

7. The breathing detection device of claim 6, wherein the power source is rechargeable and comprises a charging port mounted to a wall of the first body.

8. The breathing detection device of claim 1, wherein the temperature sensor comprises a thermistor.

9. The breathing detection device of claim 1, wherein the temperature sensor mount is formed in a shape of a bottleneck.

10. The breathing detection device of claim 1, wherein a portion of the protrusions that define the first shape taper towards one another, and wherein a portion of the protrusions that define the second shape are parallel with one another.

11. The breathing detection device of claim 10, wherein an end of the temperature sensor mount that interfaces with the cavity is sealed around the temperature sensor wiring passing therethrough such that air and other fluids are prevented from entering the cavity.

12. A tracheostomy monitoring system, comprising:
a breathing detection device, comprising:
a rigid housing comprising a first body and a second body coupled to one another,
a first tubular body extending outwards from an exterior of the first body, and
a second tubular body extending outwards from an exterior of the second body,
wherein proximal ends of the first tubular body and second tubular body interface with one another to define a channel that extends across the housing and is sized to pass air therethrough,
wherein the first body defines a cavity sized to house a plurality of electrical components comprising at least a microcontroller, a temperature sensor configured to detect a temperature of air passing through the channel, an audio sensor configured to detect a sound of the air passing through the channel, and a transceiver configured to transmit to a computing device temperature data and audio data associated with air passing through the channel, and
wherein the first body comprises a temperature sensor mount having protrusions that define a first shape to hold the temperature sensor therein such that the temperature sensor interfaces with the channel and that define a second shape to pass temperature sensor wiring into the cavity; and
a computing device comprising a memory storing instructions, a communications module, and at least one processor coupled to the communications module and the memory, the at least one processor configured to execute the instructions to:
receive temperature data and audio data associated with air passing through the channel of the breathing detection device,
determine a breath per minute (BPM) associated with each of the received temperature data and the audio data,
provide and display a first alert on the computing device based on the BPM associated with the temperature data and the audio data being outside a predetermined BPM range for a first threshold period of time,
provide and display a second alert on the computing device based on the BPM associated with the temperature data and the audio data being zero for a second threshold period of time, and
provide and display a third alert on the computing device based on the temperature data indicating a decrease in temperature for a third threshold period of time.

13. The tracheostomy monitoring system of claim 12, wherein the second body comprises a removably lid that encloses the cavity of the first body.

14. The tracheostomy monitoring system of claim 12, wherein a length of the second tubular body is smaller than a length of the first tubular body.

15. The tracheostomy monitoring system of claim 12, wherein the second tubular body is sized to fit within an opening of a heat and moisture exchanger attachment or a Passy-Muit valve attachment.

16. The tracheostomy monitoring system of claim 12, wherein the first tubular body is sized to fit over an opening of a tracheostomy tube or a pediatric tracheostomy tube adapter.

17. The tracheostomy monitoring system of claim 12, wherein the breathing detection device further comprises a rechargeable power source positioned within the cavity and comprises a charging port mounted to a wall of the first body.

18. The tracheostomy monitoring system of claim 12, wherein the temperature sensor mount is formed in a shape of a bottleneck.

19. The tracheostomy monitoring system of claim 12, wherein a portion of the protrusions that define the first shape taper towards one another, and wherein a portion of the protrusions that define the second shape are parallel with one another.

20. The tracheostomy monitoring system of claim 19, wherein an end of the temperature sensor mount that interfaces with the cavity is sealed around the temperature sensor wiring passing therethrough such that air and other fluids are prevented from entering the cavity.
